(19) United States
(12) Patent Application Publication
(10) Pub. No.: US 2003/0135115 A1
(43) Pub. Date: Jul. 17, 2003

(54) Method and apparatus for spatial registration and mapping of a biopsy needle during a tissue biopsy

(76) Inventors: Everette C. Burdette, Champaign, IL (US); Dana L. Deardorff, Oakland, CA (US)

Correspondence Address: Benjamin L. Volk, Jr., Thompson Coburn LLP, One US Bank Plaza, Suite 3500, St. Louis, MO, 63101-9928, US

(21) Appl. No.: 10/230,986

(22) Filed: Aug. 29, 2002

Related U.S. Application Data
(63) Continuation-in-part of application No. 08/897,326, filed on Jul. 21, 1997, now Pat. No. 5,993,602.
(63) Continuation-in-part of application No. 09/573,415, filed on May 18, 2000, now Pat. No. 6,512,942.
(63) Continuation of application No. 09/087,453, filed on May 29, 1998, now Pat. No. 6,129,670.
(63) Continuation-in-part of application No. 08/977,362, filed on Nov. 24, 1997, now Pat. No. 6,256,529.
(60) Provisional application No. 60/315,829, filed on Aug. 29, 2001.
(60) Provisional application No. 60/337,449, filed on Nov. 5, 2001.

(51) Int. Cl. A61B 5/05; A61B 8/00; A61B 8/12; A61B 8/14

(52) U.S. Cl. 600/437; 600/427

(57) ABSTRACT

A method for determining the location of a biopsy needle within a target volume, said target volume being defined to be a space inside a patient, the method comprising: (1) generating a plurality of images of the target volume; (2) spatially registering the images; (3) generating a three-dimensional representation of the target volume from the spatially registered images; (4) determining the location of the biopsy needle in the three-dimensional target volume representation; and (5) correlating the determined biopsy needle location with the spatially registered images. Preferably, the present invention includes graphically displaying the target volume representation, the target volume representation including a graphical depiction of the determined biopsy needle location.

### CROSS-REFERENCE AND PRIORITY CLAIM TO RELATED APPLICATIONS

This application claims the benefit under 35 U.S.C. §119(e) of provisional patent application Ser. No. 60/315,829 entitled “Method for Spatial Registration and Mapping of Tissue Biopsy”, filed Aug. 29, 2001, and provisional patent application Ser. No. 60/337,449 entitled “Apparatus and Method for Registration, Guidance and Targeting of External Beam Radiation Therapy”, filed Nov. 8, 2001, the disclosures of both of which are incorporated by reference herein.

This application is also a continuation-in-part of pending U.S. patent application Ser. No. 08/897,326, filed Jan. 14, 2002, which is a continuation of U.S. Pat. No. 6,256,529, which is a continuation-in-part of U.S. Pat. No. 6,208,883, which is a continuation of U.S. Pat. No. 5,810,007, the disclosures of all of which are incorporated by reference herein.

This application is also a continuation-in-part of pending U.S. patent application Ser. No. 09/573,415, filed May 18, 2000, which is a continuation of U.S. Pat. No. 6,129,670, which is a continuation-in-part of U.S. Pat. No. 6,256,529, which is a continuation-in-part of U.S. Pat. No. 6,208,883, which is a continuation of U.S. Pat. No. 5,810,007, the disclosures of all of which are incorporated by reference herein.

### FIELD OF THE INVENTION

The present invention relates generally to tissue biopsy procedures. More particularly, the present invention relates to a design and use of an integrated system for spatial registration and mapping of tissue biopsy procedures.

### BACKGROUND OF THE INVENTION

The concept of obtaining a tissue biopsy sample to determine whether a tumor inside the human body is benign or cancerous is conventionally known. Currently, the only clinically acceptable technique to determine whether a tumor in the human body is benign or cancerous is to extract a tissue biopsy sample from within the patient's body and analyze the extracted sample through histological and pathological examination. The tissue biopsy sample is typically obtained by inserting a biopsy needle into the tumor region and extracting a core sample of the suspected tissue from the tumor region. This procedure is often performed with real-time interventional imaging techniques such as ultrasound imaging to guide the biopsy needle and ensure its position within the tumor. The tissue biopsy process is typically repeated several times throughout the tumor to provide a greater spatial sampling of the tissue for examination.

Although moderately effective, this conventional biopsy process includes a number of limitations. For example, the conventional biopsy process is often unable to positively detect cancerous tissue that is present, also referred to as false negative detection error. The reporting of false negative results is due primarily to the limited spatial sampling of the tumor tissue; while the pathologist is able to accurately determine the malignancy of the cells in the tissue sample, undetected cancer cells may still be present in the regions of the tumor volume that were not sampled.

Furthermore, the conventional biopsy procedure does not include any spatial registration of the biopsy tissue samples to the tumor volume and surrounding anatomy. In other words, the pathology report provides the status of the tissue, but typically does not provide accurate information regarding where the tissue samples were located within the body. As a result, the clinician does not receive potentially important information for both positive and negative biopsy results.

For negative biopsy results, the spatial location of the biopsy samples would be useful for a follow-up biopsy. In such situations, it would be helpful to know the exact location of the previously tested tissue in order to select different regions within the tumor to increase the sampling area. For positive biopsies, the spatial registration information could be used to provide the clinician with a three-dimensional spatial map of the cancerous region(s) within the tissue, allowing the potential for conformal therapy that is targeted to this localized diseased region. Effectively, an anatomical atlas of the target tissue can be created with biopsy locations mapped into the tissue. This information can be used to accurately follow up disease status post-treatment. Additionally, spatial registration information could also be used to display a virtual reality three-dimensional map of the biopsy needles and samples within the surrounding anatomy in substantially real time, improving the clinician's ability to accurately sample the tissue site.

For illustrative purposes, but not limitation, one example application that would benefit from spatial registration and mapping of tissue biopsy is prostate cancer. Adenocarcinoma of the prostate is the most commonly diagnosed cancer in males in the U.S., with approximately 200,000 new cases each year. A prostate biopsy is performed when cancer is suspected, typically after a positive digital rectal examination or an elevated prostate specific antigen (PSA) test. However, it has been reported that detection of prostate cancer is missed (false negatives) in approximately 20-30% of the 600,000 men that undergo prostate biopsy in the U.S. each year—i.e. current techniques are missing over 100,000 patients of prostate cancer each year. Real time spatial registration and mapping of the biopsy tissue samples and subsequent follow-up procedures could be used to improve the rate of these false negatives by displaying more accurate information to the clinician. Furthermore, once cancer is found, a three-dimensional spatial mapping of the biopsy samples would allow for more accurate staging and treatment of the localized disease.

### SUMMARY OF THE INVENTION

In view of these and other shortcomings in the conventional tissue biopsy procedures, the inventors herein have invented a method for determining the location of a biopsy needle within a target volume, said target volume being defined to be a space inside a patient, the method comprising: (1) generating a plurality of images of the target volume; (2) spatially registering the images; (3) generating a three-dimensional representation of the target volume from the spatially registered images; (4) determining the location of the biopsy needle in the three-dimensional target volume representation; and (5) correlating the determined biopsy needle location with the spatially registered images.

The invention further may further comprise graphically displaying the target volume representation, the target volume representation including a graphical depiction of the determined biopsy needle location. Preferably, the target volume representation is graphically displayed in substantially real-time. Further still, the present invention preferably includes determining the biopsy needle location corresponding to a biopsy sample extraction, wherein the graphically displayed target volume representation includes a graphical depiction of the determined biopsy needle location corresponding to the biopsy sample extraction.

The images are preferably ultrasound images produced by an ultrasound probe. These images may be from any anatomical site that can be imaged using ultrasound and biopsied based upon that image information. In one embodiment, the ultrasound probe is preferably a transrectal ultrasound probe or a transperineal ultrasound probe. The biopsy needle is preferably inserted into the patient transrectally or transperineally. In another embodiment, the ultrasound probe is an external probe that is used to image soft tissue such as the breast for biopsy guidance.

Spatial registration is preferably achieved through the use of a localization system in conjunction with a computer. Preferably, localization uses (1) a camera disposed on the ultrasound probe at a known position and orientation relative to the ultrasound probe's field of view and (2) a reference target disposed at a known position and orientation relative to a three-dimensional coordinate system and within the camera's field of view. The reference target also includes a plurality of identifiable marks thereon having a known spatial relationship with each other. A computer receives the ultrasound image data, the camera image data, and the known positions as inputs and executes software programmed to spatially register the ultrasound images relative to each other within the target tissue volume. Disposing the camera on the probe reduces the likelihood of occlusion from disrupting the spatial registration process. However, other localization systems using frameless stereotaxy techniques that are known in the art may be used in the practice of the present invention. Further still, localization system systems other than frameless stereotaxy may be used in the practice of the present invention. An example includes a spatially-registered ultrasound probe positioning system.

Once the ultrasound images are spatially registered, the position of the biopsy needle is readily correlated thereto by the computer software. The biopsy needle position may be determined through a known spatial relationship with the ultrasound probe's field of view. Additionally, the biopsy needle position, assuming the needle is visible in at least one of the ultrasound images, may be determined through a pattern recognition technique such as edge detection that is applied to the images. Further, the ultrasound images need not be generated contemporaneously with the actual biopsy sample extraction (although it would be preferred) because the biopsy sample extraction can be guided by correlation with previously-obtained images that are spatially registered.

By providing physicians with accurate information about the location of the biopsy needle in three-dimensional space, the present invention increases the likelihood that the biopsy results will be accurate because meaningful spatial sampling can be achieved.

Further, because the positional location of each biopsy sample is accurately known, the present invention facilitates the planning process for treating any diseased portions of the target volume because additional procedures to identify the location of the diseased portion of the target volume during a planning phase of a treatment program are unnecessary. The results of the tissue biopsy (i.e. malignant vs. benign) can be displayed in 3-D space registered with the appropriate surrounding anatomy of the target volume for easy evaluation by a clinician.

Further still, providing the physician with the ability to accurately track and location a biopsy needle during a biopsy procedure allows the physician to extract biopsy samples from desired locations, such as locations that may be diagnosed as problematic through diagnostics techniques such as neural networks.

These and other features and advantages of the present invention will be in part pointed out and in part apparent upon review of the following description and the attached figures.

### BRIEF DESCRIPTION OF THE DRAWINGS

FIG. 1 is an overview of a preferred embodiment of the present invention for a transrectal prostate biopsy using a preferred frameless stereotactic localization technique;

FIG. 2 is an overview of a preferred embodiment of the present invention for a transperineal prostate biopsy using a preferred frameless stereotactic localization technique;

FIG. 3 is an overview of a preferred embodiment of the present invention for a transrectal prostate biopsy wherein a positioner/stepper is used for localization;

FIG. 4 is an overview of a preferred embodiment of the present invention for a transperineal prostate biopsy wherein a positioner/stepper is used for localization;

FIG. 5 is an example of a three-dimensional target volume representation with graphical depictions of sample locations included therein.

### DETAILED DESCRIPTION OF THE PREFERRED EMBODIMENTS

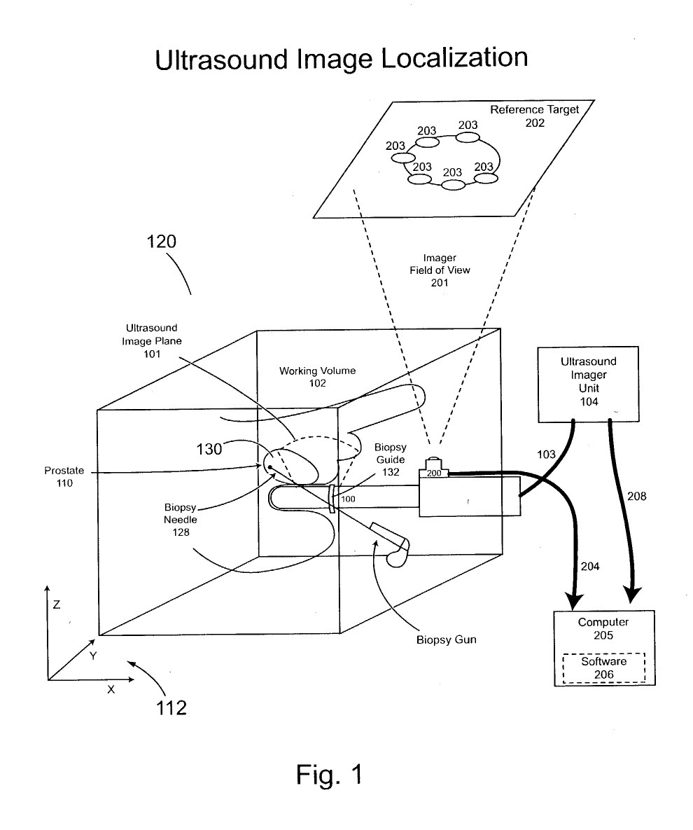

FIG. 1 illustrates an overview of the preferred embodiment of the present invention for a transrectal prostate biopsy using a preferred technique for localization. In FIG. 1, a target volume 110 is located within a working volume 102. In the invention's preferred application to prostate biopsies, the target volume 110 would be a patient's prostate or a portion thereof, and the working volume 102 would be the patient's pelvic area, which includes sensitive tissues such as the patient's rectum, urethra, and bladder. Working volume 102 is preferably a region somewhat larger than the prostate, centered on an arbitrary point on a known coordinate system 112 where the prostate is expected to be centered during the biopsy procedure. However, it must be noted that the present invention, while particularly suited for prostate biopsies, is also applicable to biopsies of other anatomical regions including but not limited to the liver, breast, brain, kidney, pancreas, lungs, heart, head and neck, colon, rectum, bladder, cervix, and uterus.

A medical imaging device 100, in conjunction with an imaging unit 104, is used to generate image data 206 corresponding to objects within the device 100's field of view 101. During a tissue biopsy procedure, the target volume 110 will be within the imaging device's field of view 101. Preferably, the medical imaging device 100 is an ultrasound probe and the imaging unit 104 is an ultrasound imaging unit. Even more preferably, the ultrasound probe 100 is a transrectal ultrasound probe or a transperineal ultrasound probe. Together, the ultrasound probe 100 and ultrasound imaging unit 104 generate a series of spaced two-dimensional images (slices) of the tissue within the probe's field of view 101. Although ultrasound imaging is the preferred imaging modality, other forms of imaging that are registrable to the anatomy, such as x-ray, computed tomography, or magnetic resonance imaging, may be used in the practice of the present invention.

It is important that the exact position and orientation of ultrasound probe 100 relative to known three-dimensional coordinate system 112 be determined. To localize the ultrasound probe to the coordinate system 112, a localization system is used.

Preferably, this localization system is a frameless stereotactic system. Even more preferably, the localization system is a frameless stereotactic system as shown in FIG. 1, wherein a camera 200 is disposed on the ultrasound probe 100 at a known position and orientation relative to the probe's field of view 101. The camera 200 has a field of view 201. A reference target 202 is disposed at some location, preferably above or below the patient examination table, in the room 120 that is within the camera 200's field of view 201 and known with respect to the coordinate system 112. Preferably, reference target 202 is positioned such that, when the probe's field of view 101 encompasses the target volume 110, reference target 202 is within camera field of view 201. Target 202 is preferably a planar surface supported by some type of floor-mounted, table-mounted, ceiling-mounted structure. Reference target 202 includes a plurality of identifiable marks 203 thereon, known as fiducials. Marks 203 are arranged on the reference target 202 in a known spatial relationship with each other.

To calibrate the camera 200 to its surroundings, the camera 200 is placed at one or more known positions relative to the coordinate system 112. When the camera 200 is used to generate an image of the reference target 202 from such known positions, the images generated thereby are be provided to computer 205. Software 206 that is executed by computer 205 includes a module programmed to identify the positions of the marks 203 in the image. The software 206 then applies a position-determination algorithm to determine the position and orientation of the camera 200 relative to the reference target 202 using, among other things, the known camera calibration positions, as is known in the art. Once the position and orientation of the camera 200 relative to the reference target 202 is known from one or more positions within the coordinate system 112, the computer 205 has calibration data that allows it to localize the position and orientation of the camera at a later time relative to the coordinate system 112. Such calibration can be performed regardless of whether the camera 200 is disposed on the probe 100. The working volume is determined by the size of the region of the field of view of the camera relative to the visibility of the active sources or or passive targets.

After calibration has been performed, the ultrasound probe 100 (with camera 200 attached thereto at a known position and orientation relative to the probe's field of view 101) can be used in “freehand” fashion with its location determined by computer 205 so long as the reference target 202 remains in the camera field of view 201. When subsequent camera images are passed to computer 205, software 206 applies similar position-determination algorithms to determine the position and orientation of the camera 200 relative to the reference target 202. By derivation, software 206 is then able to (1) determine the position and orientation of the camera 200 relative to the coordinate system 112 (because the position of the reference target 202 in coordinate system 112 is known), (2) determine the position and orientation of the probe field of view 110 relative to the coordinate system 112 (because the position and orientation of the camera 202 relative to the probe field of view 101 is known and because, as stated, the position and orientation of the camera 200 relative to the coordinate system 112 has been determined), and (3) determine the position and orientation of the content of the ultrasound image produced by the ultrasound probe 100 relative to the coordinate system 112 (because the ultrasound image contents have a determinable spatial relationship with each other and a known spatial relationship with the probe's field of view 101).

Position-determination algorithms are well-known in the art. Examples are described in Tsai, Roger Y., “An Efficient And Accurate Camera Calibration Technique for 3D Machine Vision”, Proceedings of IEEE Conference on Computer Vision and Pattern Recognition, Miami Beach, Fla., 1986, pages 364-74 and Tsai, Roger Y., “A Versatile Camera Calibration Technique for High-Accuracy 3D Machine Vision Metrology Using Off-the Shelf TV Cameras and Lenses”, IEEE Journal on Robotics and Automation, Vol. RA-3, No. 4, August 1987, pages 323-344, the entire disclosures of which are incorporated herein by reference. A preferred position-determination algorithm is an edge-detection, sharpening and pattern recognition algorithm that is applied to the camera image to locate and identify specific marks 203 on the target 202 with subpixel accuracy. Repeated linear minimization is applied to the calculated location of each identified mark 203 in camera image coordinates, the known location of each identified point in world coordinates, vectors describing the location and orientation of the camera in world coordinates, and various other terms representing intrinsic parameters of the camera. The position and orientation of the ultrasound image is computed from the position and orientation of the camera and the known geometry of the probe/camera system.

The identifiable marks 203 may be light emitting diodes (LED's) and the camera 200 may be a CCD imager. However, other types of emitters of visible or infrared light to which the camera 200 is sensitive may be used. The identifiable marks 203 may also be passive reflectors or printed marks visible to the camera 200 such as the intersection of lines on a grid, the black squares of a checkerboard, markings on the room's wall or ceiling. Any identifiable marks 203 that are detectable by the camera 200 may be used provided they are disposed in a known spatial relationship with each other. The size of the marks 203 is unimportant provided they are of sufficient size for their position within the camera image to be reliably determined.

It is advantageous for the marks 203 to be arranged in a geometric orientation, such as around the circumference of a circle or the perimeter of a rectangle. Such an arrangement allows the computer software 206 to apply known shape-fitting algorithms that filter out erroneously detected points to thereby increase the quality of data provided to the position-determination algorithms. Further, it is advantageous to arrange the marks 203 asymmetrically with respect to each other to thereby simplify the process of identifying specific marks 203. For example, the marks 203 may be unevenly spaced along a circular arc or three sides of a rectangle.

Various camera devices may be used in the practice of the present invention in addition to CCD imagers, including non-linear optic devices such as a camera having a fish-eye lens which allows for an adjustment of the camera field of view 201 to accommodate volumes 102 of various sizes. In general, a negative correlation is expected between an increased size of volume 102 and the accuracy of the spatial registration system. Also, camera 200 preferably communicates its image data 204 with computer 205 as per the IEEE-1394 standard.

Camera 200 is preferably mounted at a position and orientation on the probe 100 that minimizes reference target occlusion caused by the introduction of foreign objects (for example, the physician's hand, surgical instruments, portions of the patient's anatomy, etc.) in the camera field of view 201. Further, it is preferred that the camera 200 be mounted on the probe 100 as close as possible to the probe's field of view (while still keeping reference target 202 within camera field of view 201) because any positional and orientation errors with respect to the spatial relationship between the camera and probe field of view are magnified by the distance between the camera and probe field of view.

The number of marks 203 needed for the reference target is a constraint of the particular position-determination algorithm selected by a practitioner of the present invention. Typically a minimum of three marks 203 are used. In the preferred embodiment, six marks 203 are used. In general, the positional and orientational accuracy of the localization system increases as redundant marks 203 are added to the reference target 202. Such redundant marks 203 also help minimize the impact of occlusion.

While the localization system described above (wherein a camera is mounted on the probe and a reference target is disposed in the room) may be used in the practice of the present invention, other localization systems known in the art may also be used. For example, it is known to include identifiable marks on the probe and place the camera at a known position in the room. However, it is advantageous to place the camera on the probe and the reference target at a known position in the room because there will typically be a wider range of locations in the room that are available for disposing the reference target than there will be for disposing a camera. As such, the risk of occlusion is minimized through a greater likelihood of finding a location for the reference target that is within the camera's field of view. Further, localization systems using acoustic frameless stereotaxy (which utilizes acoustic emitters and receivers rather than light emitters/receivers) or electromagnetic frameless stereotaxy (which utilizes electromagnetic emitters and receivers rather than light emitters/receivers) may used in the practice of the present invention.

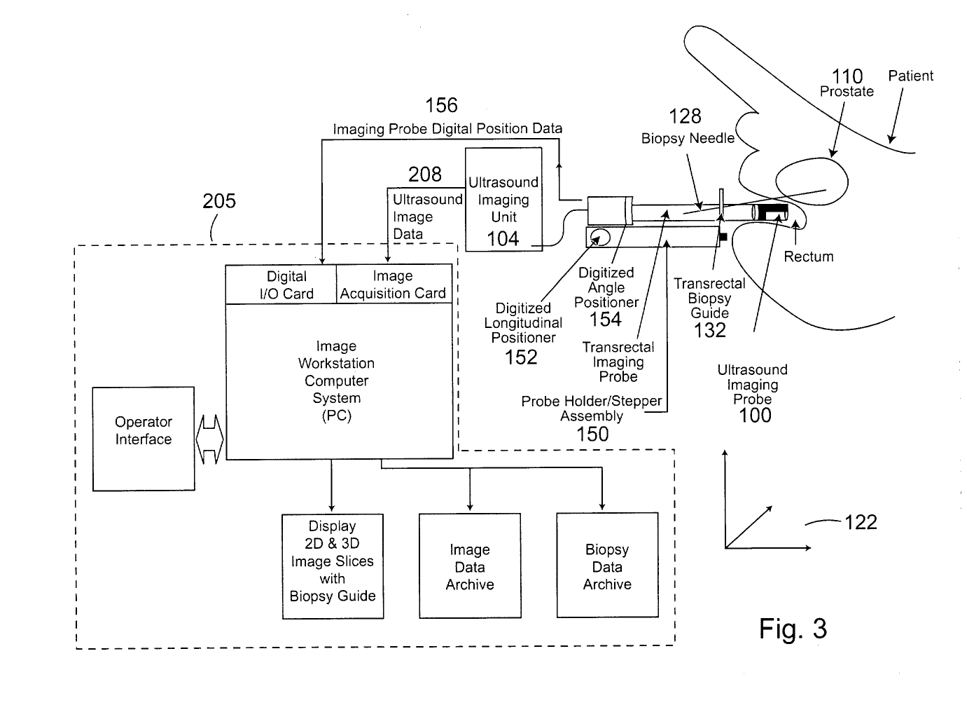

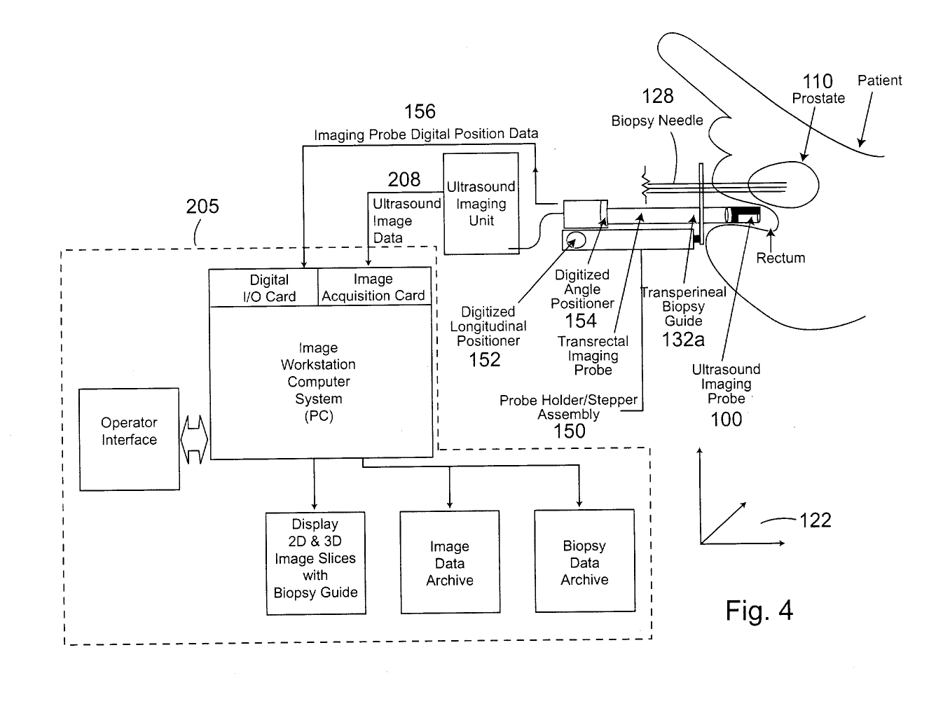

Moreover, the localization system need not use frameless stereotaxy. Localization may be achieved through other techniques known in the art such as a mechanical system that directly attaches the biopsy needle apparatus to the ultrasound probe such as a standard biopsy guide 132, a mechanical system that directly attaches the biopsy needle apparatus to the patient's body using a harness, a mechanical system that positions the imaging probe and biopsy guide with electronic spatial registration of the probe and image positions in 3D and directly attaches to the patient table or some other fixed frame of reference. Examples of such common fixed frames of reference include articulated arms or a holder assembly for the ultrasound probe and/or biopsy needle apparatus having a known position and configured with a positionally encoded stepper for moving the ultrasound probe and/or biopsy needle apparatus in known increments. FIGS. 3 and 4 illustrate examples of such a localization technique for, respectively, transrectal and transperineal prostate biopsies. In FIGS. 3 and 4, the probe 100 is disposed on a probe holder/stepper assembly 150. The probe holder/stepper assembly 150 has a known position and orientation in the coordinate system 112. A digitized longitudinal positioner 152 and a digitized angle positioner 154 are used to position the probe 100 in known increments from the assembly 150 position. The assembly 150 provides digital probe position data 156 to computer 205 which allows the computer software to determine the position and orientation of the probe in the coordinate system. An example of a suitable holder/stepper assembly can be found in _U.S. Pat. No. 6,256,529 and pending U.S. patent application Ser. No. 09/573,415, both of which being incorporated by reference herein.

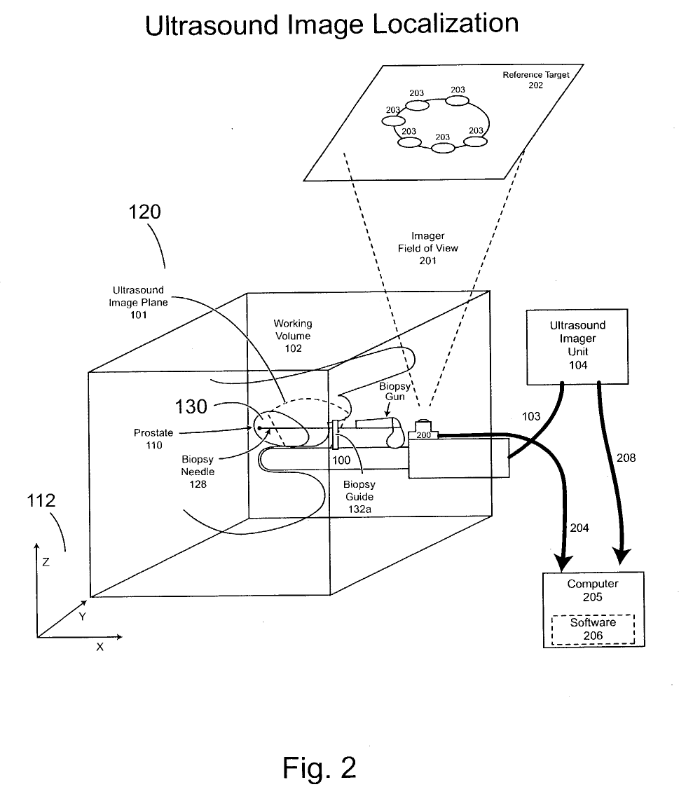

Returning to FIG. 1, biopsy needle 128 is preferably disposed in a biopsy guide 132 and inserted into the target volume 110, preferably through either the patient's rectum (FIG. 1) or perineum (FIG. 2). The physician operates the needle 128 to extract a biopsy sample from location 130 within the tumor volume. It is this location 130 that is spatially registered by the present invention.

The identification of a needle in a target volume shown in an ultrasound image is known in the art of prostate brachytherapy, as evidenced by U.S. Pat. No, 6,129,670 (issued to Burdette et al.), the entire disclosure of which is incorporated herein by reference. For example, biopsy needle 128 preferably has a known trajectory relative to the camera 200 which allows localization of the biopsy needle tip once the camera is localized. However, this need not be the case as the presence of the biopsy needle may also be independently detected within the spatially registered ultrasound images. Typically, the needle will stand out in bright contrast to the surrounding tissues in an ultrasound images, and as such, known pattern recognition techniques such as edge detection methods (camfers and others) can be used to identify the needle's location in the ultrasound images. Because the images are spatially registered, the location of the biopsy needle relative to the coordinate system is determinable.

Computer 205 records the location 130 each time a biopsy sample is extracted. The needle position at the time the biopsy sample is extracted is determined in two ways: (1) based upon the known trajectory of the needle relative to the image and the 3D volume as it is fired from the biopsy device 129 (known as a biopsy gun), and (2) based upon auto-detection of the needle in the ultrasound image as it is “fired” from the biopsy gun 129. As the ultrasound probe continues to generate images of the target volume, the needle's movement within the target volume can be tracked, and its determined location continuously updated, preferably in real-time.

The construction of a three-dimensional representation of a target volume from a plurality of ultrasound image slices is also known in the art of prostate brachytherapy, as evidenced by the above-mentioned '670 patent. Applying this technique to tissue biopsies, and enhancing that technique by depicting the spatially registered location 130 of each biopsy sample extraction in the three-dimensional representation of the target volume, a physician is provided with valuable information as to the location of previous biopsy samples within the target volume. Further, these locations 130 can be stored in some form of memory for later use during treatment or treatment planning.

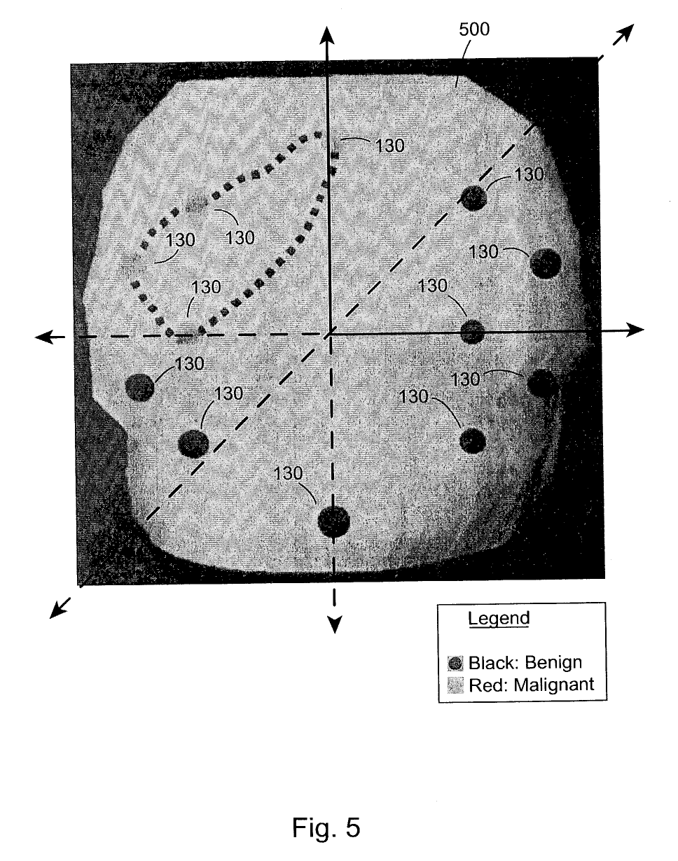

FIG. 5 illustrates an exemplary three-dimensional representation 500 of a target volume 110. The locations 130 of the biopsy sample extractions are also graphically depicted with the 3-D representation 500. Because the 3-D representation 500 is spatially registered, the three-dimensional coordinates of each biopsy sample location 130 is determinable.

As a further enhancement, once the biopsy sample has been analyzed to determine whether the tissue is malignant or benign, the present invention allows such data to be entered into computer 205. Thereafter, software 206 executes a module programmed to record the analyzed status of each biopsy sample and note that status on the three-dimensional representation of the target volume 110. For example, the software may color code the biopsy sample locations 130 depicted in the three-dimensional representation 500 with to identify the status, as shown in FIG. 5 (wherein black is used for a benign status and white is used for a malignant status-other color coding schemes being readily devisable by those of ordinary skill in the art).

The biopsy needle 128 may be attached to the ultrasound probe via a biopsy needle guide 132 as shown in FIGS. 1-4. However, this need not be the case as the biopsy needle can be an independent component of the system whose position in the ultrasound images is detected through pattern recognition techniques, as mentioned above. Another aspect of the invention is using the spatially registered images of the target volume in conjunction with a neural network to determine the optimal locations within the target volume from which to extract biopsy samples. The neural network would be programmed to analyze the spatially registered images and identify tissue regions that appear cancerous or have a sufficiently high likelihood of cancer to justify a biopsy. Because the images are spatially registered, once the neural network identifies desired locations within the target volume for extracting a biopsy sample, the physician is provided with a guide for performing the biopsy that allows for focused extraction on problematic regions of the target volume. Having knowledge of desired biopsy sample extraction locations, the physician can guide the biopsy needle to those locations using the techniques described above.

While the present invention has been described above in relation to its preferred embodiment, various modifications may be made thereto that still fall within the invention's scope, as would be recognized by those of ordinary skill in the art following the teachings herein. As such, the full scope of the present invention is to be defined solely by the appended claims and their legal equivalents.

What is claimed is:

1. A method for determining the location of a biopsy needle within a target volume, said target volume being defined to be a space inside a patient, the method comprising:
generating a plurality of images of the target volume;
spatially registering the images;
generating a three-dimensional representation of the target volume from the spatially registered images;
determining the location of the biopsy needle in the three-dimensional target volume representation; and
correlating the determined biopsy needle location with the spatially registered images.

2. The method of claim 1 further comprising graphically displaying the target volume representation, the target volume representation including a graphical depiction of the determined biopsy needle location.

3. The method of claim 2 further comprising tracking the biopsy needle location as the biopsy needle moves within the target volume through repetitive performance of the method steps.

4. The method of claim 3 further comprising graphically displaying the target volume representation in substantially real-time as the biopsy needle location is tracked.

5. The method of claim 2 wherein the image generating step comprises generating a plurality of ultrasound image slices of the target volume using an ultrasound probe having a field of view encompassing the target volume, and wherein the spatial registration step comprises localizing the position and orientation of the ultrasound probe in a three-dimensional coordinate system and determining the spatial position and orientation of the ultrasound image generated by the ultrasound probe using the localized ultrasound probe position and orientation.

6. The method of claim 5 wherein the biopsy needle location determining step comprises determining the biopsy needle location using a known spatial relationship between the biopsy needle and the ultrasound probe field of view.

7. The method of claim 5 wherein the biopsy needle is visible in at least one of the images, wherein the biopsy needle location determining step comprises determining the biopsy needle location from the spatially registered images by applying a pattern recognition algorithm to the spatially registered images.

8. The method of claim 5 wherein the ultrasound probe localizing step comprises localizing the ultrasound probe using frameless stereotaxy.

9. The method of claim 8 wherein a camera having a field of view is disposed on the ultrasound probe in a known spatial relationship with the ultrasound probe's field of view, wherein a reference target having a plurality of identifiable marks is disposed at a known position in the coordinate system within the camera's field of view, the identifiable marks having a known spatial relationship with each other, and wherein the ultrasound probe localization step comprises:
imaging the reference target with the camera;
determining the position of the camera relative to the imaged reference target using the known spatial relationship between the identifiable marks;
determining the position of the camera relative to the coordinate system using the determined camera position relative to the reference target; and
determining the position of the ultrasound probe's field of view relative to the coordinate system using the known spatial relationship between the camera and the ultrasound probe's field of view.

10. The method of claim 2 wherein the biopsy needle position determining step comprises determining the position of the biopsy needle in the three-dimensional target volume representation when a biopsy sample is extracted thereby.

11. The method of claim 2 wherein each biopsy sample has a known cancerous status, the method further comprising:
for each biopsy sample, associating its cancerous status with the determined position from which it was extracted; and
wherein graphically displaying step includes graphically displaying the cancerous status associated with each determined position depicted in the target volume representation.

12. The method of claim 2 further comprising storing the target volume registration for subsequent retrieval.

13. A system for determining the location of a biopsy needle within a target volume, said target volume being defined to be a space inside a patient, the system comprising:
an imaging device having a field of view that generates a plurality of images of the target volume;
a localization system associated with the imaging device that locates the field of view in space;
a computer programmed to (1) spatially register the plurality of images, (2) generate a three-dimensional representation of the target volume from the spatially registered images, (2) determine the location of the biopsy needle in the three-dimensional target volume representation, and (3) correlate the determined biopsy needle location with the spatially registered images.

14. The system of claim 13 wherein the computer is further programmed to graphically display the target volume representation, the target volume representation including a graphical depiction of the determined biopsy needle position.

15. The system of claim 14 wherein the computer is further programmed to track the biopsy needle location as the biopsy needle moves within the target volume.

16. The system of claim 15 wherein the computer is further programmed to graphically display the target volume representation in substantially real-time as the biopsy needle location is tracked.

17. The system of claim 14 wherein the imaging device is an ultrasound probe that generates a plurality of ultrasound image slices of the target volume, the ultrasound probe having a field of view that encompasses the target volume, and wherein the spatial registration system localizes the position and orientation of the ultrasound probe in a three-dimensional coordinate system and determines the spatial position and orientation of the ultrasound image generated by the ultrasound probe using the localized ultrasound probe position and orientation.

18. The system of claim 17 wherein the biopsy needle has a known spatial relationship between itself and the ultrasound probe field of view, and wherein the computer is further programmed to determine the biopsy needle location using its known spatial relationship with the ultrasound probe field of view.

19. The system of claim 17 wherein the biopsy needle is visible in at least one of the images, wherein the biopsy needle has a known spatial relationship between itself and the ultrasound probe field of view, and wherein the computer is further programmed to determine the biopsy needle location from its relative position within the spatially registered images using a pattern recognition algorithm.

20. The system of claim 17 wherein the localization system is a frameless stereotaxy system.

21. The system of claim 20 wherein the localization system comprises:
a camera having a field of view and disposed on the ultrasound probe in a known spatial relationship with the ultrasound probe's field of view;
a reference target disposed at a known position in the coordinate system within the camera's field of view, the reference target having a plurality of identifiable marks, the identifiable marks having a known spatial relationship with each other; and
wherein the computer is further programmed to (1) receive camera image data from the camera corresponding to an image of the reference target, (2) determine the position of the camera relative to the imaged reference target using the known spatial relationship between the identifiable marks, (3) determine the position of the camera relative to the coordinate system using the determined camera position relative to the reference target, and (3) determine the position of the ultrasound probe's field of view relative to the coordinate system using the known spatial relationship between the camera and the ultrasound probe's field of view.

22. The system of claim 14 wherein the computer is further programmed to determine the position of the biopsy needle in the three-dimensional target volume representation when a biopsy sample is extracted thereby.

23. The system of claim 14 wherein each biopsy sample has a known cancerous status, and wherein the computer is further programmed to (1) for each biopsy sample, associate its cancerous status with the determined position from which it was extracted, and (2) graphically display in the target volume representation the cancerous status associated with each determined biopsy sample position depicted therein.

24. The system of claim 14 wherein the computer is further programmed to store the target volume registration for subsequent retrieval.

25. A system for determining the location of a biopsy needle within a target volume, said target volume being defined to be a space inside a patient, the method comprising:
means for generating a plurality of images of the target volume, at least one of the images depicting the biopsy needle within the target volume;
means for spatially registering the images;
means for generating a three-dimensional representation of the target volume from the spatially registered images;
means for determining the location of the biopsy needle in the three-dimensional target volume representation; and
means for correlating the determined biopsy needle location with the spatially registered images.

26. The system of claim 25 further comprising means for graphically displaying the target volume representation, the target volume representation including a graphical depiction of the determined biopsy needle position.

27. The system of claim 26 further comprising means for tracking the biopsy needle location as the biopsy needle moves within the target volume.

28. The system of claim 27 further comprising means for graphically displaying the target volume representation in substantially real-time as the biopsy needle location is tracked.

29. The system of claim 26 further comprising means for determining the position of the biopsy needle in the three-dimensional target volume representation when a biopsy sample is extracted thereby.

30. The system of claim 29 further comprising means for determining the biopsy needle location using a known spatial relationship between the biopsy needle and a field of view of the image generating means.

31. The method of claim 29 wherein the biopsy needle is visible in at least one of the images, the method further comprising means for determining the biopsy needle location from the spatially registered images by applying a pattern recognition algorithm to the spatially registered images.

32. The system of claim 26 wherein each biopsy sample has a known cancerous status, the system further comprising:
means for associating each biopsy sample's cancerous status with the determined position from which it was extracted; and
means for graphically displaying the cancerous status associated with each determined position depicted in the target volume representation.

33. The system of claim 26 further comprising means for storing the target volume registration for subsequent retrieval.

34. A system for localizing a medical imaging device, the system comprising:
a medical imaging device having a field of view, the medical imaging device being configured to generate medical images of a target volume inside a patient's body as the target volume appears within its field of view;
a camera having a field of view and disposed on the medical imaging device in a known position and orientation relative to the medical imaging device's field of view;
a reference target disposed in a known position and orientation relative to a three-dimensional coordinate system and within the camera's field of view, the reference target having a plurality of identifiable marks thereon disposed in a known spatial relationship with each other; and
a computer configured to (1) receive medical images from the medical imaging device and camera images of the reference target from the camera, and (2) determine the position and orientation of the medical imaging device's field of view relative to the coordinate system.

35. The system of claim 34 wherein the medical imaging device is an ultrasound probe.

36. The system of claim 35 wherein the identifiable marks are light emitting diodes.

37. The system of claim 35 wherein the camera is a camera having a fisheye lens.

38. A method for localizing a medical imaging device, the method comprising:
disposing a camera on a medical imaging device, the medical imaging device having a field of view, the camera having a field of view and a known position and orientation relative to the medical imaging device's field of view;
disposing a reference target at a known position and orientation relative to a three-dimensional coordinate system and within the camera's field of view, the reference target having a plurality of identifiable marks thereon that are arranged in a known spatial relationship with each other;
generating an image of the reference target with the camera; and
determining the position and orientation of the medical imaging device's field of view relative to the coordinate system.

39. The method of claim 38 wherein the medical imaging device is an ultrasound probe.

40. A method of determining suitable locations for biopsy sample extractions, the method comprising:
generating a plurality of images of a target volume from which a biopsy sample is to be extracted;
spatially registering the images; and
applying the spatially registered images to a neural network programmed to determine from the spatially registerd images a plurality of desired biopsy sample locations within the target volume.

41. The method of claim 40 further comprising:
extracting biopsy samples from the determined desired biopsy sample locations.
